(19) United States
(12) Patent Application Publication
(10) Pub. No.: US 2025/0298000 A1
(43) Pub. Date: Sep. 25, 2025

(54) PREPARATION OF CULTURE MEDIUM AND USE THEREFOR IN WATER TOXICITY COLORIMETRIC DETECTION

(71) Applicant: CHANGCHUN INSTITUTE OF APPLIED CHEMISTRY, CHINESE ACADEMY OF SCIENCE, Changchun, Jilin (CN)

(72) Inventors: Dengbin YU, Changchun, Jilin (CN); Rongbing LI, Changchun, Jilin (CN); Shaojun DONG, Changchun, Jilin (CN)

(73) Assignee: CHANGCHUN INSTITUTE OF APPLIED CHEMISTRY, CHINESE ACADEMY OF SCIENCE, Changchun, Jilin (CN)

(21) Appl. No.: 18/852,456

(22) PCT Filed: Sep. 22, 2023
(86) PCT No.: PCT/CN2023/120615

§ 371 Date: Jun. 17, 2025

(30) Foreign Application Priority Data
Sep. 30, 2022 (CN) 202211206829.8

(51) Int. Cl. G01N 33/18; C12N 1/20; C12R 1/125; C12R 1/445; G01N 21/33; G01N 21/78

(52) U.S. Cl. CPC G01N 33/1813; C12N 1/205; G01N 21/33; G01N 21/78; G01N 33/1866; C12R 2001/125; C12R 2001/445

(57) ABSTRACT

The present invention provides a culture medium and use thereof in water toxicity colorimetric detection. The culture medium comprises glucose, peptone, NaCl, a beef extract, and a ferric salt. The solubility of the ferric salt in water is not high, such that part of the ferric salt remains resuspended in a solution along with cultured bacteria to continuously release Fe³⁺ after centrifugation and washing, thereby successfully constructing a water toxicity colorimetric detection sensor which is simple, environment-friendly, and visible to naked eyes.

This application claims the priority of Chinese Patent Application No. 202211206829.8, filed with the China National Intellectual Property Administration on Sep. 30, 2022, and titled with “PREPARATION OF CULTURE MEDIUM AND USE THEREFOR IN WATER TOXICITY COLORIMETRIC DETECTION”, which is hereby incorporated by reference in entirety.

### FIELD

The present disclosure relates to the technical field of detection methods, and specifically relates to the preparation of a culture medium and use thereof in colorimetric detection of water toxicity.

### BACKGROUND

With the rapid development of modern chemical industry and agriculture in China, a large number of chemicals have been researched, developed and used. Concomitantly, industrial wastewater, pesticides and the like are discharged into water, resulting in the pollution of rivers, streams, lakes and oceans. In recent years, China has increasingly focused its attention on the detection and monitoring of toxic and harmful pollutants, given that some pollutants in wastewater are extremely detrimental to the water environment. Article 25 of the People's Republic of China (PRC) Water Pollution Prevention and Control Law, which came into effect in 2018, stipulates that the state shall improve and strengthen the monitoring of water environment quality and water pollutant discharge. However, the low concentration and high toxicity of toxic chemicals such as heavy metals and pesticide organics in water present certain challenges to the detection of indicators such as biological toxicity.

In recent years, various methods for comprehensive toxicity detection of whole-cell water based on microorganisms have been developed one after another. Among these methods, colorimetric detection of water toxicity is favored by the majority of scientific researchers due to a visible response signal to the naked eye, the simplicity of the required detection instruments, and the low cost of detection. Nevertheless, there is a paucity of colorimetric detection methods for water toxicity. The existing methods are either overly complex or exhibit suboptimal detection efficiency when utilizing a single-channel ultraviolet detector, which presents a significant challenge in their practical implementation.

The commercial marine culture medium used in our previous research contains ferric salt with low solubility, which can still release Fe³⁺ continuously throughout the bacterial culture and subsequent centrifugation, washing and storage processes. Based on this principle, we constructed a colorimetric detection sensor for water toxicity. However, this marine culture medium is not suitable for the culture of freshwater bacteria. As a prokaryotic organism, Bacillus subtilis, a freshwater bacterium, is widely used as a detection subject for toxicity detection. According to the present disclosure, the addition of ferric salt with low solubility to the traditional CM culture medium enables the preparation of a culture medium, which is suitable for the culture of freshwater bacteria and capable of continuous Fe³⁺ release, facilitating the construction of a colorimetric detection sensor. In non-toxic and toxic conditions, the amount of reduced mediator potassium ferrocyanide generated by the reaction between Bacillus subtilis and oxidized potassium ferricyanide varies, as does the amount of Prussian blue (PB) generated by the continuous release of Fe³⁺ in the culture medium and potassium ferrocyanide. The colorimetric detection of water toxicity is conducted based on the difference in green color obtained by combining blue PB and yellow potassium ferricyanide.

The existing colorimetric detection methods for water toxicity detection have the following disadvantages: (1) the procedure for creating a composite clay is intricate; (2) the financial outlay is considerable, and glucose, fructose, or galactose, in addition to chemical reagents such as ethanol and aminopropyl triethoxysilane, are required; (3) the potassium ferricyanide and Fe³⁺ that are fixed by clay are susceptible to deterioration and loss, which impacts the detection efficacy; (4) the single-channel ultraviolet detection efficiency is low.

It is therefore imperative to develop a simple, environmentally friendly, efficient, and visible colorimetric detection method for water toxicity.

### SUMMARY

In view of this, the technical problem to be solved by the present disclosure is to provide a detection method for water toxicity, which is simple, environmentally friendly, visible to the naked eye and high in detection efficiency.

A culture medium is provided by the present disclosure, which comprises:
glucose, peptone, NaCl, beef extract and a ferric salt.

Preferably, a mass ratio of glucose, peptone, NaCl, beef extract and the ferric salt is 20:15:5:0.5:0.001-10; and
the ferric salt is selected from the group consisting of ferric citrate, ammonium ferric citrate, ferric sulfate, and a mixture thereof.

Use of the culture medium described in any one of the above in colorimetric detection of water toxicity is provided by the present disclosure.

A method for colorimetric detection of water toxicity is provided by the present disclosure, which comprises:
A) adding a suspension of bacteria, potassium ferricyanide and water to be detected into a well plate, mixing and reacting to obtain a sample to be detected;
adding a suspension of bacteria, potassium ferricyanide and standard water into a well plate, mixing and reacting to obtain a standard sample; and
B) measuring an ultraviolet absorption spectrum of the standard sample and the sample to be detected, recording a peak value of the ultraviolet absorption spectrum, and calculating an inhibition rate and/or IC₅₀ value.

Preferably, the bacteria are Bacillus subtilis and/or Staphylococcus aureus.

Preferably, the suspension of bacteria is prepared by:
culturing bacteria with shaking, centrifuging, centrifuging and washing with NaCl solution, and re-suspending in NaCl solution to obtain the suspension of bacteria;
wherein the culture is performed at 50-300 rpm at 15-40° C. for 8-30 h; the centrifugation is performed at 3000-10000 rpm for 1-20 min; and NaCl has a concentration of 0.1-30%.

Preferably, the suspension of bacteria in step A) has an OD₆₀₀ of 0.1-20; the potassium ferricyanide has a concentration of 0.5-50 mM; and the reaction is performed at a temperature of 4-45° C. for a time of 1-120 min.

Preferably, the inhibition rate is calculated as: Inhibition %=(1−Absₜₒₓ/Abs_{con})×100(1); wherein, Absₜₒₓ represents the peak value of the ultraviolet absorption spectrum of the sample to be detected at 690 nm, and Abs_{con} represents the peak value of the ultraviolet absorption spectrum of the standard sample at 690 nm; and

the IC₅₀ value is calculated by: taking a concentration of toxicant in the water to be detected as the abscissa and the inhibition rate as the ordinate, drawing a curve, and fitting to obtain the IC₅₀ value.

Preferably, the water to be detected comprises metal ions of Cd²⁺, Hg²⁺, Zn²⁺, Cr⁶⁺, U⁶⁺, Te³⁺, Co³⁺, Se⁶⁺, Pu³⁺, Hg²⁺, Mn⁴⁺, and/or Cd²⁺, organic matter of 3,5-dichlorophenol and/or formaldehyde, or a binary, ternary or multinary mixture thereof, or the water to be detected is an actual water sample;
a concentration of toxicant in the water to be detected is 0.001-200 mg·L⁻¹; and
the well plate is a 2-200 well plate.

Preferably, the measurement of the ultraviolet absorption spectrum is performed using one or more of a microplate reader, an ultraviolet spectrophotometer, a color picker software or a colorimetric card; and the measurement is to measure a peak value of the ultraviolet absorption spectrum at 690 nm or an RGB value; and
the method includes directly observing a color of the sample with naked eyes, comparing the color difference between the toxic sample and the standard sample, and determining whether the sample to be detected is toxic or the degree of toxicity; or comprises picking RGB color or electrochemically measuring a current value, obtaining an inhibition rate, and determining whether the sample to be detected is toxic or the degree of toxicity.

Compared with the prior art, the present disclosure provides a culture medium and use thereof in water toxicity detection. The culture medium comprises glucose, peptone, NaCl, beef extract and a ferric salt. The ferric salt has low solubility in water, such that a portion of the ferric salt remains resuspended in the solution along with the cultured bacteria, thereby continuously releasing Fe³⁺ after centrifugation and washing. This allows for the successful construction of a colorimetric detection sensor for water toxicity that is simple, environmentally friendly, and visible to naked eyes.

### BRIEF DESCRIPTION OF DRAWINGS

FIG. 1. (A) Ultraviolet absorption spectra and (B) sample photographs of bacteria cultured in two culture media after reacting with potassium ferricyanide; Sample 1 (11, 12, 13) is a mixed sample of bacteria cultured in CM culture medium and potassium ferricyanide, and sample 2 (21, 22, 23) is a mixed sample of bacteria cultured in CM-Fe culture medium and potassium ferricyanide.

FIG. 2. Optimization of reaction time of (A) 10 min; (B) 20 min; (C) 25 min; and (D) 30 min;

FIG. 3. Toxicity detection of wastewater containing Cu²⁺ for (A) inhibition rate curves and (B) corresponding photographs of the samples. The final concentrations of toxicants from top to bottom are: 0 mg/L, 0.49 mg/L, 0.98 mg/L, 1.95 mg/L, 3.9 mg/L, 7.8 mg/L and 15.63 mg/L respectively;

FIG. 4. Toxicity detection of wastewater containing Hg²⁺ for (A) inhibition rate curves and (B) corresponding photographs of the samples. The final concentrations of toxicants from top to bottom are: 0 mg/L, 0.25 mg/L, 0.49 mg/L, 0.98 mg/L, 1.95 mg/L, 3.9 mg/L, 7.8 mg/L and 15.63 mg/L respectively;

FIG. 5. Photographs of samples with (A) ferric citrate added to the culture medium and (B) ferric nitrate added to the culture medium;

FIG. 6. Photographs of samples with (A) 0.5 g/L ferric citrate added to the culture medium and (B) 0.0001 g/L ferric citrate added to the culture medium.

### DETAILED DESCRIPTION

The present disclosure provides the preparation of a culture medium and use thereof in colorimetric detection of water toxicity. Those skilled in the art can refer to the contents herein and appropriately improve the process parameters to achieve. It should be particularly pointed out that all similar substitutions and modifications are obvious to those skilled in the art, and they all fall within the scope of protection of the present disclosure. The method and use of the present disclosure have been described through preferred embodiments, and those skilled in the art can apparently make modifications or appropriate changes and combinations to the method and use herein without departing from the content, spirit and scope of the present disclosure to realize and apply the technology of the present disclosure.

### Prussian Blue:

Prussian blue (PB) is a common hexacyanoferrate, that is, ferric ferrocyanide with the chemical formula of Fe₄[Fe(CN)₆]₃. It is a coordination compound, which can be used for glazing and as a dye for oil paintings. PB is expected to be used in electrochromic devices, electrocatalysis and spectroelectrochemical research because of its unique electrochromic properties and reversible redox behavior.

### Culture Medium:

It refers to the nutrient substrate formulated from a combination of different nutrients which is supplied to microorganisms, plants, or animals (or tissues) for growth and reproduction.

### Colorimetry:

It is a method to determine the content of components to be detected by comparing or measuring the color shade of colored substance solution at a specific wavelength. There are two commonly used colorimetry methods: visual colorimetry and photoelectric colorimetry. The former is observed by eyes and the latter is measured by photoelectric colorimeter. Both methods are based on Beer-Lambert law (see ultraviolet-visible spectrophotometry).

### RGB Color Mode:

It is a color standard in the industry, in which the three color channels of red (R), green (G) and blue (B) are changed and superimposed to reproduce a broad array of colors. RGB represents the colors of the three channels of red, green, and blue. This standard covers almost all colors that can be perceived by human vision and is one of the most widely used color systems.

### Microplate Reader

A microplate reader, also known as an enzyme-linked immunosorbent assay (ELISA) detector, is a specialized instrument utilized in the ELISA process. It is also referred to as a microplate detector. The microplate reader can be classified into two main categories: semi-automatic and automatic. Despite their differences, these categories share a similar operational principle, with a colorimeter serving as the core component and colorimetry being the analytical technique employed. In general, the final volume of the detection solution must be less than 250 μL, which exceeds the capacity of an ordinary photoelectric colorimeter. Consequently, special requirements have been established for photoelectric colorimeters in microplate readers.

### Colorimetric Detection of Water Toxicity

The presence and the degree of toxicity of the sample are determined by comparing the color difference between the sample to be detected and the standard sample.

A culture medium is provided by the present disclosure, which comprises:
glucose, peptone, NaCl, beef extract and a ferric salt.

In some preferred embodiments of the disclosure, a mass ratio of glucose, peptone, NaCl, beef extract and the ferric salt is 20:15:5:0.5:0.001-10.

Specifically, the ferric salt is selected from the group consisting of ferric citrate, ammonium ferric citrate, ferric sulfate, and a mixture thereof.

The above mixture is ground into powder with a ball mill to obtain a new culture medium. Preferably, it is ground into powder by a dry grinding method. The ball milling in the present disclosure is preferably carried out to obtain a particle diameter of 100-2000 mesh.

The culture medium of the present disclosure generally contain various trace elements, mainly include iron, copper, zinc, selenium, manganese and the like. Iron is an essential element, because it is involved in many enzymes participating in DNA replication and cell metabolism. Iron deficiency will cause cell cycle to stagnate in G0 or G1 phase, and even lead to apoptosis of rapidly divided cells.

Use of the culture medium described in any one of the above in colorimetric detection of water toxicity is provided by the present disclosure.

According to the present disclosure, the above culture medium is not completely dissolved in water, such that a portion of the ferric salt remains resuspended in the solution along with cultured bacteria, thereby continuously releasing Fe³⁺ after centrifugation and washing. In combination with the use of microplate reader, this allows for the successful construction of an efficient colorimetric detection sensor for water toxicity that is simple, environmentally friendly, and visible to naked eyes. In the process of water toxicity detection, potassium ferricyanide is reduced by bacteria to obtain potassium ferrocyanide, which reacts with Fe³⁺ continuously released from suspended bacteria to generate PB. The green substance obtained by mixing the blue PB and the yellow potassium ferricyanide based on the principle of color mixing is used as an indicator to construct a colorimetric detection sensor for water toxicity. By mixing bacteria, potassium ferricyanide and water samples in one step, the present disclosure can achieve the purpose of simple, rapid and efficient colorimetric detection of water toxicity, which greatly simplifies the detection procedure.

A method for colorimetric detection of water toxicity is provided by the present disclosure, which comprises:
A) adding a suspension of bacteria, potassium ferricyanide and water to be detected into a well plate, mixing and reacting to obtain a sample to be detected;
adding a suspension of bacteria, potassium ferricyanide and standard water into a well plate, mixing and reacting to obtain a standard sample; and
B) measuring an ultraviolet absorption spectrum of the standard sample and the sample to be detected, recording a peak value of the ultraviolet absorption spectrum, and calculating an inhibition rate and/or IC₅₀ value.

According to the present disclosure, the suspension of bacteria is prepared by:
culturing bacteria with shaking, centrifuging, centrifuging and washing with NaCl solution, and re-suspending in NaCl solution to obtain the suspension of bacteria;
wherein the culture is performed at 50-300 rpm at 15-40° C. for 8-30 h; the centrifugation is performed at 3000-10000 rpm for 1-20 min; and NaCl has a concentration of 0.1-30%, preferably 0.1-20%.

Specifically, the bacteria are Bacillus subtilis and/or Staphylococcus aureus.

In some specific embodiments, the suspension of bacteria is more preferably prepared by:
inoculating Bacillus subtilis into sterilized CM-Fe culture medium, culturing in a constant temperature shaker at 50-300 rpm at 37° C. for 8-30 h, centrifuging at 3000-10000 rpm for 1-20 min, then centrifuging and washing with 0.1-30% NaCl solution twice to remove the culture medium, and finally resuspending Bacillus subtilis in 0.1-30% NaCl solution at 0-30° C. for later use.

Adding the suspension of bacteria, potassium ferricyanide and water to be detected into a well plate, mixing and reacting to obtain a sample to be detected.

In the present disclosure, the water to be detected comprises metal ions of Cd²⁺, Hg²⁺, Zn²⁺, Cr⁶⁺, U⁶⁺, Te³⁺, Co³⁺, Se⁶⁺, Pu³⁺, Hg²⁺, Mn⁴⁺, and/or Cd²⁺, organic matter of 3,5-dichlorophenol and/or formaldehyde, or a binary, ternary or multinary mixture thereof; or the water to be detected is an actual water sample.

The concentration of toxicants in the water to be detected is 0.001-200 mg·L⁻¹; more preferably 0.49-16 mg·L⁻¹.

The well plate is a 2-200 well plate; according to the present disclosure, a 96-well plate is preferably utilized, which can measure 96 samples at the same time, and has a higher detection efficiency than the single sample measurement in the prior art

The suspension of bacteria has an OD₆₀₀ of 0.1-20, more preferably 0.1-15, most preferably 1-10. The potassium ferricyanide has a concentration of 0.5-50 mM, more preferably 2-20 mM;

The reaction temperature is 4-45° C., more preferably 10-40° C. The reaction time is 20-90 min, more preferably 25-30 min.

In a specific embodiment, OD₆₀₀=6, and the concentration of potassium ferricyanide is 8 mM.

After the bacteria cultured in the culture medium of the present disclosure react with potassium ferricyanide, an obvious absorption peak appears.

Adding a suspension of bacteria, potassium ferricyanide and standard water into a well plate, mixing and reacting to obtain a standard sample;

In order to add samples at the same time as much as possible, a multi-channel pipette is used to add samples. After the reaction, directly observing a color of the sample with naked eyes, comparing the color difference between the toxic sample and the standard sample, and determining whether the sample to be detected is toxic or the degree of toxicity.

Measuring an ultraviolet absorption spectrum of the standard sample and the sample to be detected, recording a peak value of the ultraviolet absorption spectrum, and calculating an inhibition rate and/or IC₅₀ value.

The present disclosure records the peak value of the ultraviolet absorption spectrum at 690 nm.

Three parallel experiments were carried out on all samples, and all colorimetric and electrochemical experiments are carried out at room temperature.

The inhibition rate is specifically calculated as: Inhibition %=(1−Absₜₒₓ/Abs_{con})×100(1);
wherein, Absₜₒₓ represents the peak value of the ultraviolet absorption spectrum of the sample to be detected at 690 nm, and Abs_{con} represents the peak value of the ultraviolet absorption spectrum of the standard sample at 690 nm;
the IC₅₀ value is calculated by: taking a concentration of toxicant in the water to be detected as the abscissa and the inhibition rate as the ordinate, drawing a curve, and fitting to obtain the IC₅₀ value.

The measurement of the ultraviolet absorption spectrum is performed using one or more of a microplate reader, an ultraviolet spectrophotometer, a color picker software or a colorimetric card; and the measurement is to measure a peak value of the ultraviolet absorption spectrum at 690 nm or an RGB value;
the method includes directly observing a color of the sample with naked eyes, comparing the color difference between the toxic sample and the standard sample, and determining whether the sample to be detected is toxic or the degree of toxicity; or comprises picking RGB color or electrochemically measuring a current value, obtaining an inhibition rate, and determining whether the sample to be detected is toxic or the degree of toxicity.

According to the present disclosure, the measurement of the ultraviolet absorption spectrum is performed using one or more of a microplate reader, an ultraviolet spectrophotometer, a color picker software or a colorimetric card.

The present disclosure provides a method for preparing a culture medium and use thereof in colorimetric detection of water toxicity. The new culture medium is prepared by adding ferric salt with low solubility. The ferric salt has low solubility in water, such that a portion of the ferric salt remains resuspended in the solution along with the cultured bacteria, thereby continuously releasing Fe³⁺ after centrifugation and washing. This allows for the successful construction of a colorimetric detection sensor for water toxicity that is simple, environmentally friendly, and visible to naked eyes.

The method of the present disclosure offers a simplified detection procedure, eliminating the need for clay preparation and reducing the number of required steps to a single mixing of the cultured bacteria, potassium ferricyanide, and the water sample for reaction. Additionally, the cost is relatively low because no glucose and additional chemical reagents such as ethanol and aminopropyl triethoxysilane are required. Furthermore, the method is environmentally friendly. The concentration of potassium ferricyanide is low, and a good colorimetric detection effect can be achieved by optimizing the concentration of potassium ferricyanide to only 10 mM.

In order to further illustrate the present disclosure, the preparation of a culture medium and use thereof in colorimetric detection of water toxicity provided by the disclosure are described below in detail in conjunction with examples.

### Example 1: Comparison Between the Comparative Culture Medium and the Culture Medium of the Present Disclosure

### (1) Preparation of the Culture Medium (CM-Fe) of the Present Disclosure

20 g of glucose, 15 g of peptone, 5 g of NaCl, 0.5 g of yeast extract and 0.001-10 g of ferric citrate were weighed, mixed and ground into powder by a dry grinding method in a ball mill to obtain a new culture medium CM-Fe.

### Formula of Comparative Medium CM

20 g of glucose, 15 g of peptone, 5 g of NaCl, 0.5 g of yeast extract. In addition, according to empirical values, NaOH was used to adjust the pH of the culture medium to 7.4 (a pH suitable for the growth of the currently used prokaryotic expression strain Bacillus subtilis).

### (2) Preparation of Liquid Culture Medium

1-100 g of CM and 1-100 g of CM-Fe culture media were added into two 1 L beakers respectively, adjusted to a volume of 1 L by adding deionized water, stirred, ultrasonicated, dissolved, and aliquoted in six 500 mL triangular flasks (3 for each culture medium), which were sealed with a sterile filtration sealing film, sterilized with high-temperature and high-pressure at 121° C. (98 kPa) for 5-40 min, and cooled down for later use.

### (3) Bacterial Culture

Bacillus subtilis was inoculated into sterilized CM and CM-Fe culture media respectively, and cultured in a constant temperature shaker at 50-300 rpm at 37° C. for 8-30 h. The cultured bacterial liquid was centrifuged at 3000-10000 rpm for 1-20 min, then centrifuged and washed twice with 0.1-30% NaCl solution to remove the culture medium. Finally, Bacillus subtilis was resuspended in 0.1-30% NaCl solution and placed at 0-30° C. for later use.

### (4) Comparison of the Reaction Between Bacteria Cultured in Two Culture Media and Potassium Ferricyanide

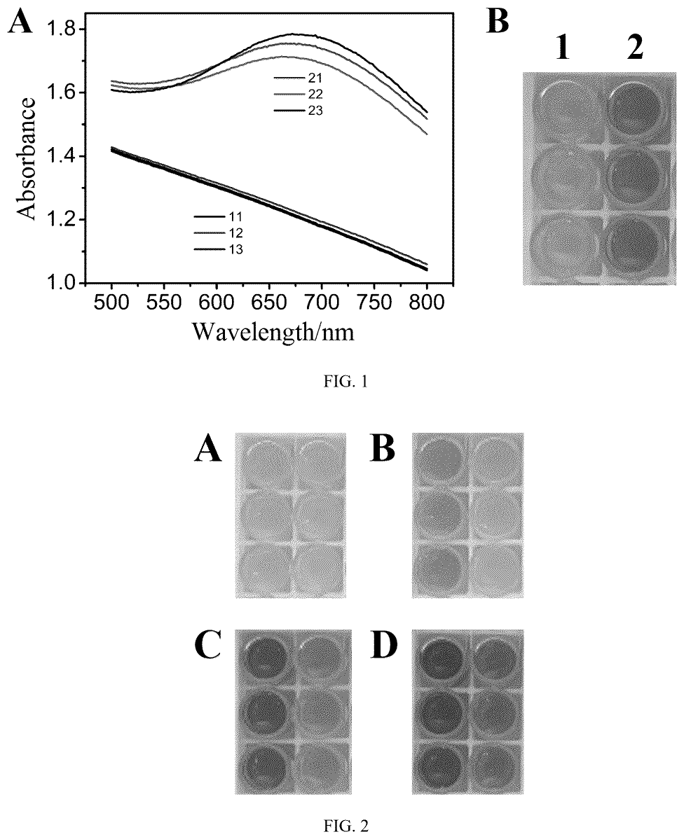

100 μL of two suspensions of bacteria with the concentration of OD₆₀₀=6, 50 μL of deionized water and 50 μL of 8 mM potassium ferricyanide were mixed in the wells of a 96-well plate and reacted at 25° C. for 20 min. FIG. 1 shows (A) ultraviolet absorption spectra and (B) sample photographs of bacteria cultured in two culture media after reacting with potassium ferricyanide. Sample 1 (11, 12, 13) is a mixed sample of bacteria cultured in CM culture medium and potassium ferricyanide, and sample 2 (21, 22, 23) is a mixed sample of bacteria cultured in CM-Fe culture medium and potassium ferricyanide.

As shown in FIG. 1A, there was no obvious absorption peak after the reaction of Bacillus subtilis cultured in CM medium with potassium ferricyanide, while an obvious absorption peak appeared after the reaction of the bacteria cultured in CM-Fe medium added with iron salt with potassium ferricyanide. It can also be clearly seen from FIG. 1B that the colors of bacteria cultured in the two culture media are obviously different after reacting with potassium ferricyanide.

### Example 2: Optimization of Reaction Time

The experimental process is similar to that in Example 1. 100 μL of a cultured suspension of bacteria with OD₆₀₀=8, 50 μL of 2 mg/L Hg²⁺, and 50 μL 20 mM potassium ferricyanide were mixed in the wells of a 96-well plate, and deionized water was used as a standard sample, for 10 min, 20 min, 25 min and 30 min of reaction respectively. As can be clearly seen from FIG. 2A, the colors of the standard sample and the sample to be detected were almost the same after 10 min of reaction. After 20 min of reaction, the color of the standard sample turned slightly green, but with little difference with the color of the sample to be detected (FIG. 2B). After 25 min of reaction, the difference between them increased (FIG. 2C). After 30 min of reaction, although the color of the sample to be detected was also deep, the color difference between them was small (FIG. 2D). Therefore, 25 minutes was the best reaction time. FIG. 2 shows the optimization of reaction time: (a) 10 min; (B) 20 min; (C) 25 min; and (D) 30 min.

### Example 3: Toxicity Detection of Wastewater Containing Cu²⁺

(1) Preparation of CM-Fe culture medium, formulation of liquid medium and bacterial culture were the same as in Example 1.
(2) Toxicity detection

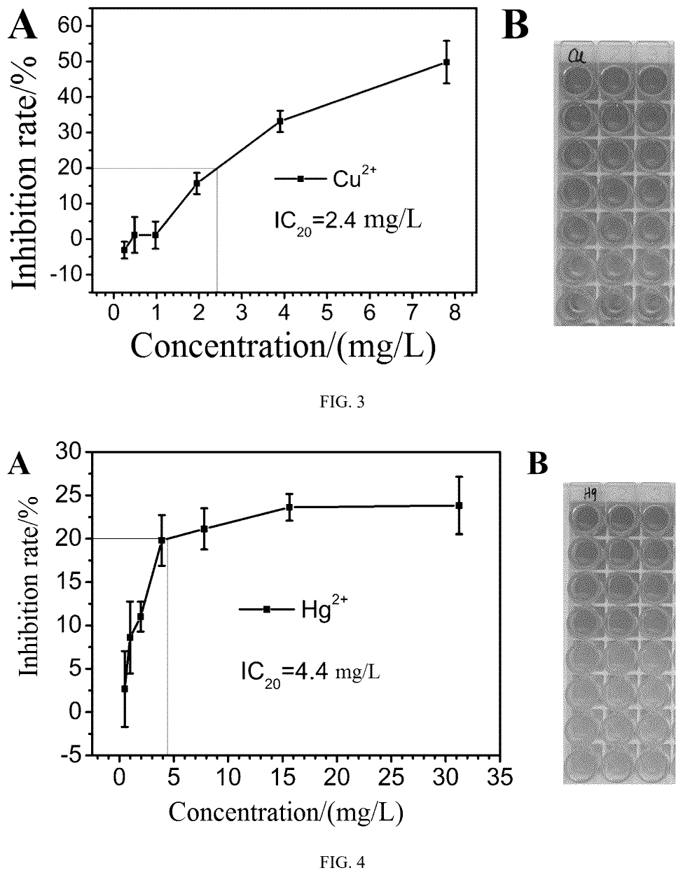

100 μL of two suspensions of bacteria with OD₆₀₀=2, 50 μL of Cu²⁺ at varying concentrations and 50 μL of 8 mM potassium ferricyanide were mixed in the wells of a 96-well plate and reacted for 20 min. The peak value of the ultraviolet absorption spectrum at 690 nm was recorded for each spectrum, and the inhibition rate curves for each concentration of toxicant were calculated according to equation (1) (FIG. 3A) and fitted to obtain IC₂₀=2.4 mg/L. The corresponding photographs of the samples are presented in FIG. 3B, which clearly demonstrates the color difference of bacteria containing toxicants with different concentrations after reacting with potassium ferricyanide. At low Cu²⁺, concentrations, the color of the toxic samples was similar to that of the non-toxic samples. At elevated Cu²⁺, concentrations, the toxic sample basically did not turn green, but only showed the yellow color of potassium ferricyanide. Samples containing 3.9 mg/L Cu²⁺ can be discerned with naked eyes, indicating that the detection limit was 3.9 mg/L. FIG. 3 shows toxicity detection of wastewater containing Cu²⁺ for (A) inhibition rate curves and (B) corresponding photographs of the samples. The final concentrations of toxicants from top to bottom were 0 mg/L, 0.49 mg/L, 0.98 mg/L, 1.95 mg/L, 3.9 mg/L, 7.8 mg/L and 15.63 mg/L, respectively.

Subsequent to a period of culture, potassium ferricyanide in the solution containing the standard water sample was reduced by microorganisms, resulting in the formation of potassium ferrocyanide. The continuous release of Fe³⁺ in the solution reacted with potassium ferrocyanide and then led to the generation of PB, which was compounded with excessive yellow potassium ferricyanide to obtain green. However, when the water sample contained toxicants, the toxicants inhibited the activity of microorganisms, resulting in a decrease in the amount of potassium ferrocyanide reduced by microorganisms. Consequently, the amount of PB generated by the reaction of Fe³⁺ continuously released in the solution with potassium ferrocyanide was correspondingly reduced. Additionally, the green color obtained by the combination of blue PB and excessive yellow potassium ferricyanide became lighter.

### Example 4: Toxicity Detection of Hg²⁺ in Water

(1) Preparation of CM-Fe culture medium, formulation of liquid culture medium and bacterial culture were the same as in Example 1.
(2) Toxicity detection

The experimental process was similar to that of Example 3, except that the toxicant was Hg²⁺ instead of Cu²⁺. FIG. 4A shows the inhibition rate curve, and FIG. 4B shows corresponding photographs of the samples, from which it can be seen that the detection limit was 0.49 mg/L.

FIG. 4 shows toxicity detection of wastewater containing Hg²⁺ for (A) inhibition rate curves and (B) corresponding photographs of the samples. The final concentrations of toxicants from top to bottom were 0 mg/L, 0.25 mg/L, 0.49 mg/L, 0.98 mg/L, 1.95 mg/L, 3.9 mg/L, 7.8 mg/L and 15.63 mg/L, respectively.

### Comparative Example 1: Comparison of Basic Culture Media Added with Ferric Citrate and With Ferric Nitrate

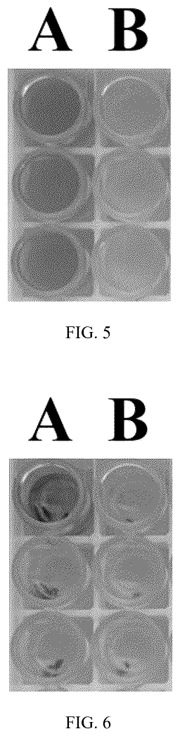

Two ferric salts were added to the basic culture medium containing glucose, peptone, NaCl and beef extract to obtain different new culture media: CM-Fe (1) obtained by adding ferric citrate and CM-Fe (2) obtained by adding ferric nitrate. Bacillus subtilis was inoculated and cultured in the two culture media. After centrifugation, washing, and resuspension, 20 mM potassium ferricyanide was added. It can be observed that the samples with the bacteria cultured in the culture medium CM-Fe (1) obtained by adding ferric citrate turned green after adding potassium ferricyanide (FIG. 5A), while the samples with the bacteria cultured in CM-Fe (2) did not turn green after adding potassium ferricyanide (FIG. 5B). FIG. 5 shows photographs of samples with (A) ferric citrate added to the culture medium and (B) ferric nitrate added to the culture medium.

### Comparative Example 2: Comparison of Basic Culture Media Added with Different Amounts of Ferric Citrate

Different amounts of ferric citrate were added to the basic culture medium containing glucose, peptone, NaCl and beef extract to obtain different new culture media: CM-Fe (3) obtained by adding 0.5 g/L ferric citrate and CM-Fe (4) obtained by adding 0.0001 g/L ferric citrate. Bacillus subtilis was inoculated and cultured in the two culture media. After centrifugation, washing, and resuspension, 10 mM potassium ferricyanide was added. It can be observed that the samples with the bacteria cultured in the culture medium CM-Fe (3) turned green after adding potassium ferricyanide (FIG. 6A), while the samples with the bacteria cultured in CM-Fe (4) did not turn green after adding potassium ferricyanide (FIG. 6B). FIG. 6 shows photographs of samples with (A) 0.5 g/L ferric citrate added to the culture medium and (B) 0.0001 g/L ferric citrate added to the culture medium.

The above embodiments are only preferred embodiments of the present disclosure. It should be noted that, for those skilled in the art, several improvements and modifications may be further made without departing from the principle of the present disclosure, and these improvements and modifications should also be deemed as falling into the scope of protection of the present disclosure.

1. A culture medium, comprising:
glucose, peptone, NaCl, beef extract and a ferric salt.

2. The culture medium according to claim 1, wherein a mass ratio of glucose, peptone, NaCl, beef extract and the ferric salt is 20:15:5:0.5:0.001-10; and
the ferric salt is selected from the group consisting of ferric citrate, ammonium ferric citrate, ferric sulfate, and a mixture thereof.

3. A method for colorimetric detection of water toxicity, comprising using the culture medium according to claim 1.

4. A method for colorimetric detection of water toxicity in water, comprising:
A) adding a suspension of bacteria, potassium ferricyanide and water to be detected into a well plate, mixing and reacting to obtain a sample to be detected;
adding a suspension of bacteria, potassium ferricyanide and standard water into a well plate, mixing and reacting to obtain a standard sample; and
B) measuring an ultraviolet absorption spectrum of the standard sample and the sample to be detected, recording a peak value of the ultraviolet absorption spectrum, and calculating an inhibition rate and/or IC₅₀ value.

5. The method according to claim 4, wherein the bacteria are Bacillus subtilis and/or Staphylococcus aureus.

6. The method according to claim 4, wherein the suspension of bacteria is prepared by:
culturing bacteria with shaking, centrifuging, centrifuging and washing with NaCl solution, and re-suspending in NaCl solution to obtain the suspension of bacteria;
wherein the culture is performed at 50-300 rpm at 15-40° C. for 8-30 h; the centrifugation is performed at 3000-10000 rpm for 1-20 min; and NaCl has a concentration of 0.1-30%.

7. The method according to claim 4, wherein the suspension of bacteria in step A) has an OD₆₀₀ of 0.1-20; the potassium ferricyanide has a concentration of 0.5-50 mM; and
the reaction is performed at a temperature of 4-45° C. for a time of 1-120 min.

8. The method according to claim 4, wherein the inhibition rate is calculated as: Inhibition %=(1−Absₜₒₓ/Abs_{con})×100(1); wherein, Absₜₒₓ represents the peak value of the ultraviolet absorption spectrum of the sample to be detected at 690 nm, and Abs_{con} represents the peak value of the ultraviolet absorption spectrum of the standard sample at 690 nm; and
the IC₅₀ value is calculated by: taking a concentration of toxicant in the water to be detected as the abscissa and the inhibition rate as the ordinate, drawing a curve, and fitting to obtain the IC₅₀ value.

9. The method according to claim 4, wherein the water to be detected comprises metal ions of Cd²⁺, Hg²⁺, Zn²⁺, Cr⁶⁺, U⁶⁺, Te³⁺, Co³⁺, Se⁶⁺, Pu³⁺, Hg²⁺, Mn⁴⁺, and/or Cd²⁺, organic matter of 3,5-dichlorophenol and/or formaldehyde, or a binary, ternary or multinary mixture thereof; or
the water to be detected is an actual water sample;
a concentration of toxicant in the water to be detected is 0.001-200 mg·L⁻¹; and
the well plate is a 2-200 well plate.

10. The method according to claim 4, wherein the measurement of the ultraviolet absorption spectrum is performed using one or more of a microplate reader, an ultraviolet spectrophotometer, a color picker software or a colorimetric card; and the measurement is to measure a peak value of the ultraviolet absorption spectrum at 690 nm or an RGB value; and
the method includes directly observing a color of the sample with naked eyes, comparing the color difference between the toxic sample and the standard sample, and determining whether the sample to be detected is toxic or the degree of toxicity; or comprises picking RGB color or electrochemically measuring a current value, obtaining an inhibition rate, and determining whether the sample to be detected is toxic or the degree of toxicity.
